(19) United States
(12) Patent Application Publication
(10) Pub. No.: US 2010/0023031 A1
(43) Pub. Date: Jan. 28, 2010

(54) NEURAL GUIDE

(76) Inventors: Francesco Catalano, Roma (IT); Antonio Merolli, Rome (IT)

Correspondence Address: Steinfl & Bruno, 301 N Lake Ave Ste 810, Pasadena, CA, 91101, US

(21) Appl. No.: 12/440,135

(22) PCT Filed: Sep. 7, 2007
(86) PCT No.: PCT/IB07/53612

§ 371 Date: Sep. 9, 2009

(30) Foreign Application Priority Data
Sep. 8, 2006 (IT) RM2006A000474

(51) Int. Cl. A61B 17/00

(52) U.S. Cl. 606/152

(57) ABSTRACT

A neural guide (1) apt to receive a pair of nerve stumps (N1, N2) to allow nerve regeneration therethrough, comprising a first (2) and a second half-shell member (3) closeable the one on the other so as to define therebetween a longitudinal seat (4) having a cylindrical geometry, said seat having a widened central section (43) for nerve regeneration and two spherical side sections (44, 45) for making the gluing points

The present invention refers to a neural guide, or neuroguide, i.e. to a device apt to promote regeneration of a nerve, in particular a peripheral nerve, therethrough.

In the last few years there has taken hold a technique for regenerating nerves, mostly peripheral ones, based on the use of tubular structures commonly referred to as neural guides or “neuroguides”. According to such a technique, the two torn or severed nerve stumps are arranged in correspondence of the longitudinal ends of the neuroguide and inserted into the internal lumen of the latter. Nerve portions adjacent to the stumps are then sutured with thread and/or fibrin glue to the neuroguide.

However, to date this surgical technique entails remarkable drawbacks, risking to thwart its clinical potential.

First of all, stump insertion into the guide proves difficult, since a perfect matching of diameters between nerve branch and guide lumen is not always attained. Moreover, this operation has to be carried out without the option of a visual checking, as evidently the internal lumen of the guide itself is not visible, and anyhow not accessible from the outside.

Moreover, thread suturing on the guide is invasive, as it entails the surgical lesion of the epineurium and the entailed chance of undesired fibroblast migration that may hinder nerve regeneration. From this standpoint, gluing techniques—both current and yet to be proposed ones—should constitute an actual progress; yet, a perfect carrying out thereof in an actual surgical field is difficult as in this case as well there can be had no visual access to the internal lumen of the guide to check for correct glue distribution.

Again, the plurality of anatomical applications would require a corresponding plurality of guide diameters to allow an optimal carrying out of the technique and avoid slacks or friction/forcing of the nerve into the guide itself, yet evidently this is not compatible with the costs of production lines for tubular guides.

Therefore, the technical problem set and solved by the present invention is to provide a neural guide allowing to overcome the drawbacks mentioned above with reference to the known art.

Such a problem is solved by a neural guide according to claim 1.

Preferred features of the present invention are set forth in the dependent claims thereof.

The present invention provides several relevant advantages. The main advantage lies in that the provision of a guide structure having two members that can be spaced the one from the other allows the surgeon to insert in an extremely simple and rapid manner the neural ends into the guide, visually checking all insertion steps. Analogously, the surgeon may also visually check step-by step the correct making of any gluing points.

Moreover, such advantages allow to overcome the drawback linked to the need of different diameters of the neuroguide, as even with a lower number of diameters the nerve insertion and locking technique may be optimally carried out or, vice versa, the production of a high number of diameters will be less complicated and expensive at an industrial level.

Other advantages, features and the operation modes of the present invention will be made apparent from the following detailed description of some embodiments thereof, given by way of example and not for limitative purposes. Reference will be made to the figures of the annexed drawings, wherein:

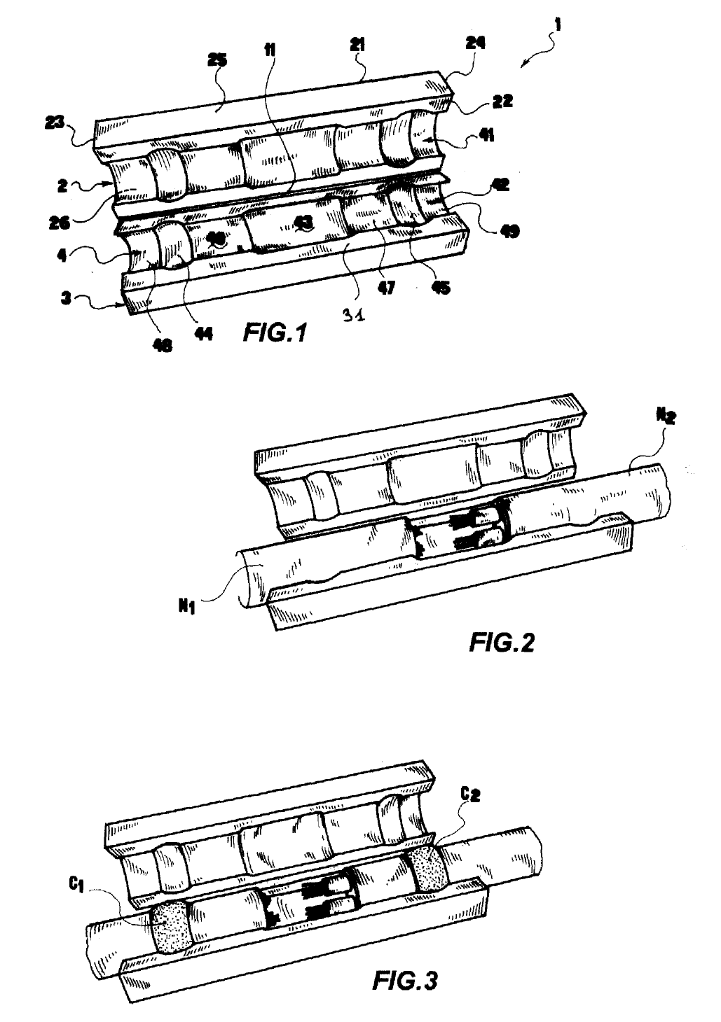

FIG. 1 shows a perspective view of a preferred embodiment of the neural guide according to the present invention;

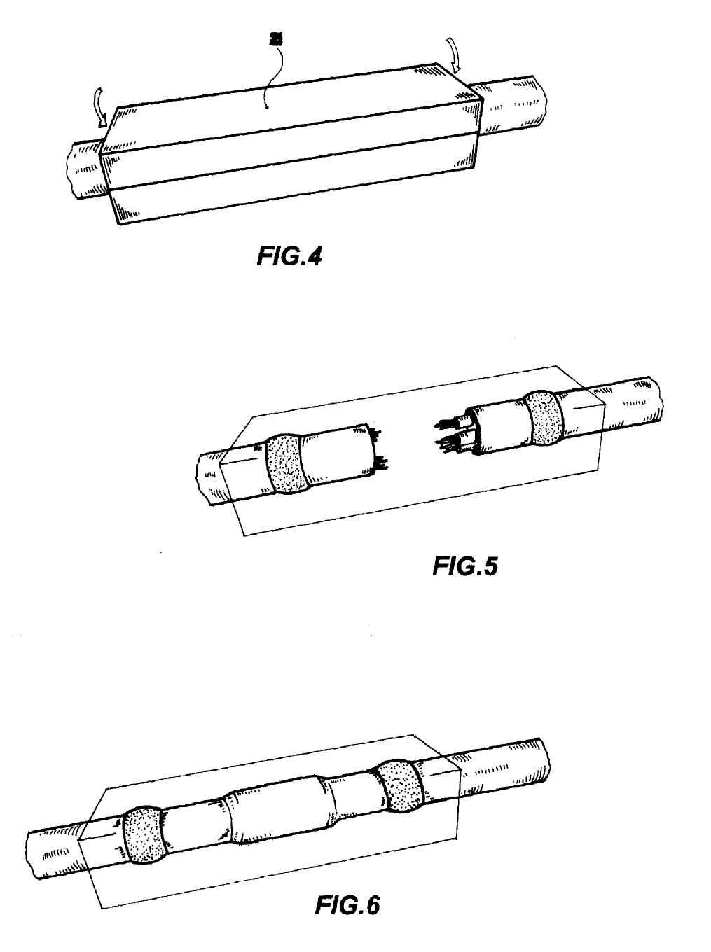

FIGS. 2 to 4 show each a perspective view of the neural guide of FIG. 1, during a respective step of the surgical use thereof; and

FIGS. 5 and 6 show partially see-through perspective views of the neural guide of FIG. 1; FIG. 5 illustrates by way of example a respective phase of the regeneration of a nerve housed into the guide itself, and FIG. 6 illustrates the gap that the guide reserves to nerve regeneration.

Referring initially to FIGS. 1 to 4, a neural guide according to a preferred embodiment of the invention is generally denoted by 1.

In the present example the neuroguide 1 has a substantially box-like structure and is formed by a first and a second member, 2 and 3 respectively, closeable the one on the other and in particular rotatably connected to each other in correspondence of a longitudinal edge 11 of the neuroguide 1 itself.

According to a preferred variant embodiment, such two members 2 and 3 closeable the one on the other are completely detachable the one from the other, instead of being permanently and/or rotatably connected. In that case—and actually also in the embodiment providing a connection of the two members 2 and 3—the device preferably comprises restraining means, like, e.g., one or more pins and respective seats, to mutually lock the two members in a closed configuration. Said variant in which the two members 2 and 3 are completely detachable is advantageous from a productive standpoint.

The first member 2 is substantially parallelepiped-shaped, comprising a top wall 21 and a bottom wall 22, parallel and having wider extension, a pair of parallel side walls 23 and 24, a front wall 25 and a rear wall 26 parallel to the front wall 25.

In correspondence of the bottom wall 22, the member 2 defines a first semi-seat 41 with a substantially semi-cylindrical geometry developing along the entire longitudinal extension of the bottom wall 22 itself.

The second member 3 has a shape analogous to that of the first member, defining, in correspondence of a top wall 31 thereof facing on the bottom wall 22 of the first member 2, a second longitudinal half-seat 42.

Therefore, the overall configuration is such that, when the first member 2 is closed on the second member 3, the two half-seats 41 and 42 lie facing the one onto the other forming an elongate longitudinal seat 4 having a substantially cylindrical geometry and extending across the neuroguide 1 itself. Therefore, in such a closed configuration the neuroguide 1 has a tubular structure and a substantially parallelepiped-shaped external shape.

Hence, the overall configuration of each of the two members 2 and 3 may be defined as a substantially half-shell one.

Analyzing now in more detail the shape of the seat 4, this has a widened regeneration section 43 apt to promote just nerve regeneration therethrough. Such a section 43 has a substantially cylindrical geometry and is centrally arranged. According to a preferred embodiment, said section has a substantially ellipsoidal geometry.

Moreover, the seat 4 has two side sections, 44 and 45 respectively, them also having a widened section and each having a substantially spherical geometry. Each of the sections 44 and 45 is apt to receive an adhesive for fixing the nerve stump therein.

With regard to the remaining sections of the seat 4, this has two substantially cylindrical sections, 46 and 47 respectively, each arranged in correspondence of a respective side of the central regeneration section 43 and interposed between the latter and a respective fixing seat 44, 45, and two further substantially cylindrical sections, 48 and 49 respectively, each arranged sideways to a respective fixing section 44, 45 and extending in correspondence of a respective longitudinal end of the seat 4 itself.

Advantageously, among its different sections 43-49 the seat 4 may have one or more transition sections having rounded corners so as not to injure the nerve housed into the seat 4 itself.

The different sections of the seat 4 may be obtained with known machining techniques, e.g. by microetching.

Of course, the neuroguide 1 is made of a biocompatible and optionally non-permeable material.

The operation mode of the neuroguide 1 will now be illustrated in detail with reference to FIGS. 2 to 4.

As mentioned above, the neural guide 1 is apt to house into the seat 4 a proximal nerve stump and a distal nerve stump, exemplarily depicted and denoted by N1 and N2 in FIG. 2, to allow their regeneration therethrough.

With a suitable distal closure the neuroguide of the invention may be used as a blind-ended or blind guide for regeneration of the sole proximal stump.

As it is shown in FIG. 2, for nerve stump insertion the neuroguide 1 is brought into an open first operating configuration, shown in FIGS. 2 and 3, in which the two members 2 and 3 are rotated with respect to each other (or separate in the above-mentioned variant embodiment) to move them away and allow access to said seat 4, and in particular to the half-seat 42 of the second member 3. Then, the two stumps are inserted from opposite sides of the seat 4, so that their ends be facing each other in correspondence of the regeneration section 43.

Once such an insertion has been carried out, as shown in FIG. 3 the nerve stumps are locked into the seat 4 by two gluing points, obtained each in correspondence of a respective fixing section 44, 45 of the seat 4 and denoted by C1 and C2. By now, it will have been better appreciated that the widened and substantially spherical shape of said sections 44 and 45 allows to optimally make said gluing points.

Moreover, to the surgeon it is offered the option of filling the seat 4 by utilizing means going from a mere saline to self-assembling peptides.

Then, the neuroguide 1 may be brought to a second closed configuration in which members 2 and 3 are rotated so as to abut to each other, housing nerve stumps therebetween.

It will be understood that the present invention is susceptible of several embodiments alternative to the hereto-described one, some of which will briefly be illustrated hereinafter with reference to the sole aspects differentiating it from the hereto-considered first embodiment.

First of all, the neuroguide may be configured so as to receive a single nerve stump, therefore being blind, as already mentioned above.

It will be appreciated that the invention also provides a novel surgical method aimed at the regeneration of peripheral nerves, providing the use of a neuroguide as described hereto and the steps illustrated referring to FIGS. 2 to 4.

The present invention has been hereto described with reference to preferred embodiments thereof. It is understood that other embodiments might exist, all falling within the concept of the same invention, and all comprised within the protective field of the claims hereinafter.

1. A neural guide adapted to receive at least one nerve stump to allow its regeneration therethrough, comprising a first member and a second member defining an internal longitudinal seat of the guide and movable with respect to each other so that the guide may assume a first open configuration in which said first member and second member are moved away to allow insertion of the at least one nerve stump into said seat, and a second closed configuration, in which said first member and second member are neared to each other, closing said seat.

2. The neural guide according to claim 1, having a substantially box-like structure.

3. The neural guide according to claim 1, wherein said longitudinal seat extends across the guide, the guide having, in said closed configuration, a substantially tubular structure.

4. The neural guide according to claim 1, wherein said longitudinal seat is a blind seat, opened only in correspondence of a longitudinal end of the guide.

5. The neural guide according to claim 1, wherein said first member and second member are each substantially in the form of a half-shell.

6. The neural guide according to claim 1, wherein said first member and second member are rotatably connected to each other.

7. The neural guide according to claim 1, wherein said first member and second member are completely detachable from each other.

8. The neural guide according to claim 1, wherein said first member and second member have each a substantially parallelepiped shape.

9. The neural guide according to claim 1, wherein said longitudinal seat has a substantially cylindrical geometry.

10. The neural guide according to claim 1, wherein said longitudinal seat comprises at least one widened fixing section, adapted to receive an adhesive to lock the at least one nerve stump thereinto.

11. The neural guide according to claim 10, wherein said seat comprises a pair of widened fixing sections, each section adapted to receive an adhesive to lock the at least one or a respective nerve stump thereinto.

12. The neural guide according to claim 10, wherein said at least one widened fixing section has a substantially spherical geometry.

13. The neural guide according to claim 10, wherein said at least one widened fixing section is arranged sideways on the seat.

14. The neural guide according to claim 1, wherein said longitudinal seat has a widened regeneration section adapted to promote nerve regeneration therethrough.

15. The neural guide according to claim 14, wherein said widened regeneration section has a substantially cylindrical geometry.

16. The neural guide according to claim 14, wherein said widened regeneration section has a substantially ellipsoidal geometry.

17. The neural guide according to claim 14, wherein said widened regeneration section is arranged in a substantially central position on the seat.

18. The neural guide according to claim 14, wherein said widened regeneration section has a microetched surface.

19. The neural guide according to claim 14, wherein said widened regeneration section it adapted to house nerve regeneration aiding preparations.

20. The neural guide according to claim 1, the neural guide being adapted to receive a pair of nerve stumps, insertable from opposite sides of said longitudinal seat.
